(19) United States
(12) Patent Application Publication
(10) Pub. No.: US 2023/0329645 A1
(43) Pub. Date: Oct. 19, 2023

(54) SYSTEM AND METHOD FOR CORRELATING OXIMETER MEASUREMENTS WITH BLOOD PRESSURE

(71) Applicant: Guy P. Curtis, San Diego, CA (US)

(72) Inventor: Guy P. Curtis, San Diego, CA (US)

(21) Appl. No.: 18/336,987

(22) Filed: Jun. 17, 2023

Related U.S. Application Data
(63) Continuation-in-part of application No. 17/496,052, filed on Oct. 7, 2021.
(60) Provisional application No. 63/172,270, filed on Apr. 8, 2021.

(51) Int. Cl. A61B 5/00; A61B 5/022; A61B 5/0205; A61B 5/1455; A61B 5/026

(52) U.S. Cl. CPC A61B 5/7246; A61B 5/022; A61B 5/0205; A61B 5/14551; A61B 5/0261; A61B 2560/0223

(57) ABSTRACT

A system and method for using an oximeter that monitors a patient's blood pressure over an extended time duration requires creating a line graph. In detail, the line graph is created to provide a steady state correlation between blood flow measurements taken by the oximeter and blood pressure measurements taken by a sphygmomanometer. To create this graph, blood pressure measurements (sphygmomanometer) and blood flow measurements (oximeter) are recorded together and collated during a heart muscle cycle of the patient. Specifically, these measurements are considered together during the same heart muscle cycle while the patient is either standing, sitting, or reclining. This establishes three respective data sets which are then used as reference points to create the line-graph. Thereafter, blood flow measurements with the oximeter can be referenced to the line-graph for direct indications of blood pressure.

This continuation-in-part application claims the benefit of U.S. Patent Application Publication No. US 2022/0328178A1, filed Oct. 7, 2021. The entire contents of application Ser. No. 17/496,052 are hereby incorporated by reference herein.

### FIELD OF THE INVENTION

The present invention pertains to systems and methods for continuously monitoring a patient's blood pressure over an extended time duration. More specifically, the present invention pertains to systems and methods that use a line graph to calibrate blood flow measurements from an oximeter with correlated blood pressure measurements from a sphygmomanometer. The present invention is particularly, but not exclusively, useful for providing continuous blood pressure information using only blood flow measurements from an oximeter.

### BACKGROUND OF THE INVENTION

For any health care situations there are many possible reasons why there is a need to measure and monitor a patient's blood pressure. Typically, this is done by intermittently using a sphygmomanometer. Although it is well known that a sphygmomanometer is a useful and reliable medical device for measuring a patient's blood pressure, its repetitive use may not be practical when continuous monitoring of a patient's blood pressure for extended time durations is necessary or preferable.

It is also well known in the medical arts that an oximeter is capable of continuously measuring blood flow. Specifically, oximeters provide measurements of blood oxygen saturation levels that are indicative of the volume of blood being measured. Oximeter measurements, however, like blood pressure measurements, are pulsatile. The respective pulses, however, have different dimensional characteristics. In the context of the present invention, the similarities and differences in the relationship between blood flow and blood pressure are important for several reasons. The similarities include:
Blood flow is a direct temporal consequence of blood pressure variations produced during a heart muscle cycle (i.e. pulse).
The dimensional characteristics of blood flow and blood pressure have concurrence in that blood pressure affects blood flow repetitively during each heart muscle cycle.
   On the other hand, for measurement purposes, there are distinctive differences between blood pressure and blood flow that must be reconciled. These differences include:
The maximum amplitude of a blood pressure measurement, “Pₘₐₓ”, and the maximum amplitude of a flood flow measurement “Fₘₐₓ” during a heart muscle cycle are inversely proportional.
“Pₘₐₓ” and “Fₘₐₓ” occur at different times during a heart muscle cycle.
The respective rates of change for “ΔPₘₐₓ” and “ΔFₘₐₓ” from pulse to pulse may be different, i.e. “ΔPₘₐₓ”≠“ΔFₘₐₓ”.
A sphygmomanometer does not measure both systolic and diastolic pressures during the same heart muscle cycle.

For reasons set for the above it is an object of the present invention to continuously recalibrate blood pressure measurements with corresponding blood flow measurements. This is done so an oximeter can be used alone, to continuously monitor blood pressure trends for successive heart muscle functions over a predetermined time duration. Another object of the present invention is to incorporate a line graph in a device which can be used to calibrate blood pressure trends with an oximeter. Yet another object of the present invention is to provide a device for measuring blood pressure with an oximeter that is easy to manufacture, is simple to use and is cost effective.

### SUMMARY OF THE INVENTION

In accordance with the present invention, a system and method are disclosed for using blood flow measurements from a patient as indications of the patient's blood pressure trends. Structurally, the system of the present invention is a combination of components that includes a sphygmomanometer, an oximeter, and a collator. Functionally, the sphygmomanometer is used to periodically measure a patient's blood pressure “P”. On the other hand, the oximeter is used continuously to measure the patient's blood oxygen saturation levels as indicators of his/her blood flow “F”. The collator then collects the blood pressure measurements and the blood flow measurements and combines selected dimensional aspects of these measurements into data sets.

For each data set, the sphygmomanometer measures a maximum blood pressure measurement “P_{systolic}” near the beginning of each heart muscle cycle. The sphygmomanometer also measures a “P_{diastolic}” during the same heart muscle cycle. In a separate operation, the oximeter measures blood flow variations that include a maximum amplitude “Fₘₐₓ” near the end of each heart muscle cycle. Further, the sphygmomanometer can also be used to measure a heart pulse rate from the patient to establish the duration for a heart muscle cycle.

As appreciated by the present invention, “P” (blood pressure) and “Fₘₐₓ” (blood flow) have an inverse relationship that must be considered in the context of a heart muscle cycle. To account for this fact, the collator collects “P_{systolic}”, “P_{diastolic}” and “Fₘₐₓ” measurements during each heart muscle cycle. From these measurements, “P_{systolic}” and “P_{diastolic}” are combined to establish a data set that can then be used as a steady state quantified “Δₛₛ”. Mathematically, “Δₛₛ”=“P_{systolic}”−“P_{diastolic}” and it is constant. Although, blood pressure changes “ΔP_{systolic}” and “P_{diastolic}” will not be directly equal to the blood flow changes “ΔFₘₐₓ”, the quantified “Δ_{SS}” for “P_{systolic}” and “P_{diastolic}” for blood pressures can be considered concurrent with “ΔFₘₐₓ”.

It happens that in a sequence of heart muscle cycles, from one heart muscle cycle to the next, “P_{diastolic}” is more reliable for use as a reference point than is “P_{systolic}”. Thus, for an operation of the present invention “P_{diastolic}” values are preferably used as reference points along a line-graph for a sequence of respective “Δₛₛ”. Functionally, the resultant line-graph can then be used for an extended time period to calibrate “Fₘₐₓ” measurement from the oximeter with a blood pressure “P” from “Δₛₛ”.

In detail, a line-graph is created for the present invention using at least two reference points. Importantly, each reference point is separately created with a “P_{systolic}”, a “P_{diastolic}” measurement which are combined in a data set for use as a quantified “Δₛₛ” at a location on the line-graph. Further, each quantified “Δₛₛ” is individually established when the patient is posed in different positions. Consequently, the plurality of quantified “Δₛₛ” can create the line-graph, with each location along the line graph providing a unique comparison “Δₛₛ”. Thus, the line graph calibrates an “F” measurement from the oximeter with a correlated “P”. With this calibration, the result is that the correlated “P” can be shown on a display as an indication of blood pressure.

As noted above, several factors must be considered during the creation of a line graph. For instance, “Pₘₐₓ” and “Fₘₐₓ” are measured separately, and they have an inverse relationship. Furthermore, between different quantified “Δₛₛ”, the rate of change “ΔPₘₐₓ” is not equal to the rate of change in “ΔFₘₐₓ”. Thus, each unique steady state comparison Δₛₛ along the line graph will change and have a new value that accounts for the fact that in a sequence each “Δₛₛ”=(P±ΔP) and (F±ΔF).

A methodology for the present invention requires a sequence of steps for obtaining blood flow measurements from a patient which can be continuously monitored and used as real time indications of the patient's blood pressure. Further, the methodology provides instructions that are useful for manufacturing a device in accordance with the present invention. It is also useful for subsequently monitoring a patient's blood pressure with the device.

In use, a sphygmomanometer is positioned on a patient to measure his/her blood pressure “P”. At the same time, an oximeter is also positioned on the patient to measure blood flow “F”. With this oximeter/sphygmomanometer combination a maximum blood pressure “Pₘₐₓ” is measured by the sphygmomanometer, and a contemporary maximum blood flow “Fₘₐₓ” is measured by the oximeter. A pulse rate measurement can also be obtained from the sphygmomanometer and be used to determine the time duration for the patient's heart muscle cycle.

Because “Pₘₐₓ” and “Fₘₐₓ” have concurrence in the same heart muscle cycle, the measured values for “Pₘₐₓ” and “Fₘₐₓ” can be collated together as components for use as a same data set. Each collated data set is thereby combined into a steady state quantified comparison “Δₛₛ”. Importantly, each quantified comparison “Δₛₛ” is unique with blood pressure and blood flow measurements. More specifically, each quantified comparison “Δₛₛ” includes measurements that are taken from the patient while he/she is posed in different positions, such as standing, sitting, or lying down.

A line graph for the present invention is created using the “Pₘₐₓ” and “Fₘₐₓ” values taken for successive quantified comparisons “Δₛₛ”. Specifically, “F” will establish the horizontal axis of the line graph, while “P” will establish the vertical axis. Because “Pₘₐₓ” and “Fₘₐₓ” have an inverse relationship, the horizontal axis of the line graph will show a decreasing value for “F”. On the other hand, the vertical axis of the line graph will show an increasing value for “P”. With this inverse relationship, each location on the resulting line graph, between quantified comparisons “Δₛₛ”, will represent a specific comparison “Δₛₛ” having unique values for “P” and “F”.

It is important to note that between any two quantified comparisons “Δₛₛ”, at each location on the line graph, the rate of change “ΔP” is not equal to the rate of change in “ΔF”. Consequently, they must be considered separately for each successive comparison “Δₛₛ”. Accordingly, values for a successive “Δₛₛ”, using values from its predecessor “Δₛₛ”, will equal (P±ΔP) and (F±ΔF). When using a line graph as disclosed here, values for “F” which are being continuously measured by an oximeter, can be directly correlated at every location along the line graph with a corresponding “P” from the same comparison “Δₛₛ”.

Additional considerations for using the methodology of the present invention include the fact that a quantified comparison “Δₛₛ” can be periodically recalibrated with updated “Pₘₐₓ” measurements taken by the sphygmomanometer (e.g. every 30 minutes). Furthermore, depending on the number of multiple quantified comparisons “Δₛₛ” that are measured, they can all be collectively used as different reference points to create a continuous line graph with differently oriented line segments (e.g. a 3-point line graph). For example, a 3-point line graph can be created having two different line segments. In this case, each line segment will be established between only two different quantified comparisons “Δₛₛ”.

### DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 shows the structural elements of a system for the present invention being used in an intended operational environment;

FIG. 2 shows respective traces of blood pressure and blood flow measurements taken during a sequence of consecutive time durations;

FIG. 3 shows the inverse relationship between blood pressure and blood flow measurements that are used to create a quantified comparison “Δₛₛ”; and

FIG. 4 is a 3-point line graph created using three distinctively different quantified comparisons “Δₛₛ”.

### DESCRIPTION OF THE PREFERRED EMBODIMENTS

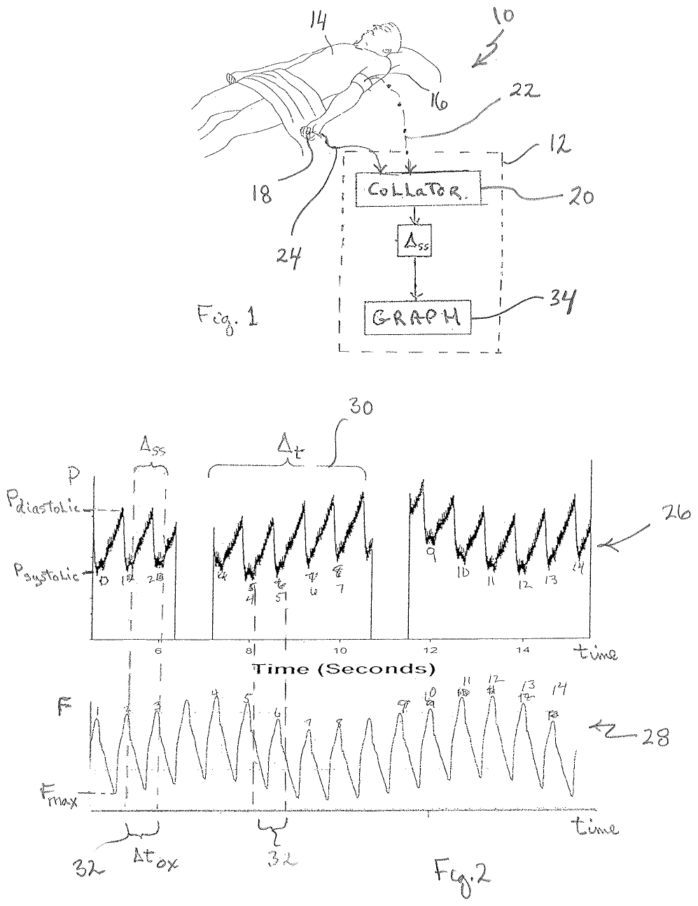

Referring initially to FIG. 1, a system for continuously using blood flow measurements “F” from a patient as indications of the patient's blood pressure “P” is shown and is generally designated 10. As shown in FIG. 1, system 10 includes an electronic device 12 that is connected directly with the patient 14. Specifically, a sphygmomanometer 16 is fitted onto the patient 14 to obtain blood pressure measurements “P” from the patient 14. Similarly, an oximeter 18 is fitted onto the patient 14 to obtain blood flow measurements “F” from the patient 14. Both measurements, “P” and “F” are taken during a same quantified comparison “Δₛₛ”.

In detail, FIG. 1 shows that the sphygmomanometer 16 is connected directly with the collator 20 of the device 12. This connection is shown in FIG. 1 as a dot-dash line 22 to signify that blood pressure measurements “P” are taken intermittently. The minor interruptions between “P” measurements are primarily due to time limitations and the labor-intensive nature for an operation of the sphygmomanometer 16. On the other hand, FIG. 1 also shows that the connection between the oximeter 18 and the collator 20 of the device 12 is a solid line 24. In this case, the solid line 24 signifies that blood flow measurements “F” from the oximeter 18 can be taken continuously with minimal, if any, interruption. In this operation, it is to be appreciated that the collator 20 functions to collect blood pressure measurements “P” along with blood flow measurements “F”. During this operation, collator 20 also functions to then combine the “P” and “F” measurements for comparison purposes.

In accordance with the present invention, the combination of a single “P” measurement and a single “F” measurement constitute a data set which is then quantified. For the present invention, quantification of the data set specifically requires creation of a steady state quantified comparison “Δₛₛ” for the measurements. As disclosed below in detail, a plurality of quantified comparisons “Δₛₛ” are required for an operation of the system 10.

Referring now to FIG. 2, the data required to establish a quantified comparison “Δₛₛ” requires “P_{systolic}” and “P_{diastolic}” that are respectively shown in a blood pressure data trace generally designate 26. Also important is the value for “Fₘₐₓ” as shown in a blood flow data trace generally designated 28. For the system 10, a common nexus between “P_{diastolic}” and “Fₘₐₓ” is shown for an exemplary episode 30 from traces 26 and 28 which last for the same time duration “Δₜ” which is the time required for a sphygmomanometer to record “Δₛₛ”. Specifically, “Δₜ” of episode 30 involves the time required for a sphygmomanometer 16 to measure at least one “P_{diastolic}” in a sequence of heart muscle cycles. Importantly, it has been determined that “P_{diastolic}” is more reliable than “P_{systolic}” as a reference for identifying “P” over extended time periods.

As shown in the blood flow trace 28 of FIG. 2, an episode 32 can be selected from within an episode 30. Specifically, a measured “P_{diastolic}” and a single a “Fₘₐₓ” will occur together only once during “Δₜ” of the episode 30. On the other hand, occurrences of “Fₘₐₓ” occur continuously for each heart muscle cycle during “Δₜ”.

In detail it happens that “Δₜ” will typically extend through several heart muscle cycles. The consequence here is that because of the operational requirements of a sphygmomanometer 16, the time interval between the “P_{diastolic}” measured in one heart cycle and “P_{diastolic}” that can be measured for the next heart muscle cycle will necessarily be delayed “Δₜ”. Although “Δₜ” will last for a few heart muscle cycles, there is only one “P_{diastolic}” that can be measured during an episode 30.

As more specifically shown in the flow data trace 28 of FIG. 2, it happens during any episode 30 for the sphygmomanometer 16, several separate episodes 32 will occur sequentially for the oximeter 18. Importantly, within the time duration “Δtₒₓ” of each episode 32 there will always be both a “Pₘₐₓ” and an “Fₘₐₓ”.

FIG. 2 also shows that although only one “P_{diastolic}” can be measured somewhere within the time duration “Δₜ” both this “Pₘₐₓ” and an “Fₘₐₓ” will occur at least once in a same episode 30 during “Δₜ”. Thus, for purposes of system 10, the measurements of “Pₘₐₓ” and “Fₘₐₓ” can be effectively considered to be concurrent. Accordingly, they can be used as components for establishing a quantified “Δₛₛ”.

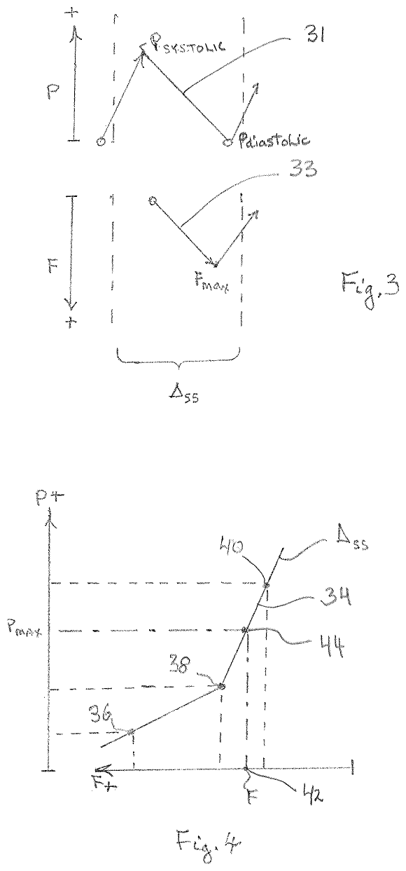

FIG. 3 shows blood pressure variations 31 for “P”, and blood flow variations 33 for “F” during a quantified “Δₛₛ”. Note: in FIG. 3 the value of “P” variations 31 increases in an upward direction. At the same time, the value of “F” variations 33 increases in a downward direction. This happens because, with an increased volume of blood flow “F”, light absorption also increases. However, with increased light absorption, the magnitude of light signals measured by an oximeter 18 are decreased. Thus, the inverse relationship. A compensation of this inverse relationship by the collator 20, which uses any “F” and only a measured “P_{diastolic}” during an episode 30, is referred to here as a quantified comparison “Δₛₛ”. For purposes of the present invention, quantified comparisons “Δₛₛ” are essential for creating a line graph 34 such as disclosed below with reference to FIG. 4.

As seen in FIG. 4, a line graph 34 is shown which constitutes a continuous sequence of comparisons “Δₛₛ”. In detail, the line graph 34 is established between quantified comparisons “Δₛₛ” which are respectively located at reference points 36, 38 and 40. All locations along the line graph 34, as well as locations on extensions therefrom beyond the points 36 and 40, each identify a unique “F” and “P” relationship for a unique “Δₛₛ”. For example, consider a measured value for “F” from the oximeter 18 which is shown at point 42. This point 42 references a point 44 on graph line 34 that calibrates “F” to a value for “P”. It is this value for “P” that corresponds with a unique “Δₛₛ” is observed by a patient 14 as his/her blood pressure.

While the particular System and Method for Correlating Oximeter Measurements with Blood Pressure as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for continuously using blood flow measurements “F” from a patient as indications of the patient's blood pressure “P”, which comprises:
a sphygmomanometer for measuring blood pressure variations in a patent's vasculature including a maximum blood pressure measurement “P_{systolic}” near the beginning of each heart muscle cycle and a pressure measurement “P_{diastolic}” near the end of each heart muscle cycle;
an oximeter for measuring blood flow variations commensurate with the blood flow variations “F” including a maximum amplitude “Fₘₐₓ” near the end of each heart muscle cycle; and
a collator connected with the sphygmomanometer and with the oximeter to establish a steady state quantified comparison “Δₛₛ” between “P_{systolic}” and “P_{diastolic}”; and
a line-graph created by a plurality of quantified comparison “Δₛₛ” for calibrating the use of measured “F” from the oximeter as an indicator of blood pressure “P” for the patient.

2. The system of claim 1 wherein the collator is preprogrammed with input information, including a heart pulse rate from the patient for identifying a duration for a heart muscle cycle, and wherein the collator collects a value for “P” relative to a value of “F” during a same heart muscle cycle to establish a data set for use in providing quantified comparison “Δₛₛ”.

3. The system of claim 2 wherein the line-graph is created with at least two reference points, wherein each reference point is identified by a separate quantified comparison “Δₛₛ”, wherein each quantified comparison “Δₛₛ” is established when the patient is respectively posed in different positions, wherein the plurality of quantified comparison “Δₛₛ” collectively establish the line-graph, and wherein each location along the line graph between quantified comparison “Δₛₛ” provides a unique “Δₛₛ” to correlate a measured “Fₘₐₓ” along the line graph, with a corresponding “P” to be indicated by a display as an indication of blood pressure.

4. The system of claim 3 wherein “Pₘₐₓ” and “Fₘₐₓ” have an inverse relationship, and further wherein between successive comparison “Δₛₛ” on the line-graph remains constant but the rate of change “ΔP” is not equal to the rate of change in “ΔF”, with a new steady state comparison “Δₛₛ” for the subsequent data set having a new value wherewith “Δₛₛ”=(P±ΔP) and (F±ΔF).

5. The system of claim 4 wherein a “P_{systolic}”, a “P_{diastolic}” and an “Fₘₐₓ” are periodically re-measured for each quantified comparison “Δₛₛ”, and wherein a re-measurement is accomplished at least every thirty minutes to reconfigure the line-graph.

6. The system of claim 4 wherein data sets are created with the patient posed standing, sitting, and lying down to respectively create the quantified comparisons “Δₛₛ” needed for a 3-point line graph.

7. The system of claim 1 wherein the duration of a heart muscle cycle is determined using blood pressure variations measured by the sphygmomanometer.

8. The system of claim 1 wherein the line graph is created using the “P” and “Fₘₐₓ” values taken for successive quantified comparisons “Δₛₛ”, and wherein to account for “P” and “Fₘₐₓ” having an inverse relationship, a horizontal axis for the graph will show a decreasing value for “Fₘₐₓ” while a vertical axis for the graph will show an increasing value for “P”, with each location on the resulting line graph between any two quantified comparisons “Δₛₛ” representing a specific comparison “Δₛₛ” having unique values for “P” relative to “Fₘₐₓ”.

9. A method for using blood flow measurements from a patient as indications of blood pressure, which comprises the steps of:
positioning a sphygmomanometer on a patient to measure blood pressure “P” of the patient, wherein “P” includes a “P_{systolic}” and a “P_{diastolic}”;
positioning an oximeter on a patient to measure blood flow “F” of the patient including an “Fₘₐₓ”;
obtaining a pulse rate measurement from the sphygmomanometer;
using the pulse rate to determine a time duration for a heart muscle cycle;
taking “P” and “Fₘₐₓ” from the measuring step for use as components in a data set wherein “P” and “Fₘₐₓ have concurrence in the same heart muscle cycle;
establishing different data sets, wherein each data set is specific with the patient posed in different positions for each data set;
quantifying each data set as an individually specific steady state quantified comparison “Δₛₛ”, wherein “P” and “Fₘₐₓ” are taken with the patient posed in different positions during the establishing step, and wherein “P” and “Fₘₐₓ” have an inverse relationship;
creating a line graph with a plurality of steady state quantified comparisons “Δₛₛ”, wherein each location on the line graph between quantified “Δₛₛ” is a unique comparison “Δₛₛ”, and further wherein between successive quantified comparisons “Δₛₛ” is constant but the rate of change “ΔP” is not equal to the rate of change in “ΔF” with a new value for each unique comparison “Δₛₛ”=(P±ΔP) and (F±ΔF);
calibrating a measured “F” with a corresponding “P” in a comparison “Δₛₛ” for every location along the line graph;
displaying “P” as an indication of blood pressure based on the graph line location for “Δₛₛ” fixed by the measured “Fₘₐₓ”.

10. The method of claim 9 wherein the data sets are periodically remeasured with updated “Pₘₐₓ” measurements taken by the sphygmomanometer and updated “Fₘₐₓ” measurements taken by the oximeter.

11. The method of claim 9 wherein “P” is measured during the heart muscle cycle, and “Fₘₐₓ” is measured near the end of the heart muscle cycle.

12. The method of claim 11 wherein “P” and “Fₘₐₓ” have concurrence within a same heart muscle cycle.

13. The method of claim 9 wherein different data sets are established with the patient respectively sitting, standing, and lying down.

14. The method of claim 13 wherein the different data sets establish a 3-point line graph.

15. The method of claim 9 wherein the line graph is created using the “P” and “Fₘₐₓ” values taken for successive quantified comparison “Δₛₛ”, and wherein to account for “P” and “F” having an inverse relationship, a horizontal axis for the line graph will show a decreasing value for “F” while a vertical axis for the graph will show an increasing value for “P”, with each location on the line graph representing a comparison “Δₛₛ” having unique values for “P” and “F” between any two quantified comparisons “Δₛₛ”.

16. A method for using blood flow measurements “F” from a patient as indications of blood pressure “P” for the patient which comprises the steps of:
measuring a blood pressure “P₁”, wherein “P₁” includes “P_{systolic1}” and “P_{diastolic1}”, and a maximum blood flow value “Fₘₐₓ₁” during a same heart muscle cycle to establish a data set therewith, wherein the data set is a first steady state quantified comparison “Δₛₛ₁”=“Pₘₐₓ₁” and “Fₘₐₓ₁”;
measuring a blood pressure “P₂”, wherein “P₂” includes “P_{systolic2}” and “P_{diastolic2}”, and a maximum blood flow value “Fₘₐₓ₂” during a same heart muscle cycle to establish a data set therewith, wherein the data set is a second steady state quantified comparison “Δₛₛ₂”=“Pₘₐₓ₂” and “Fₘₐₓ₂”;
creating a line graph using “Δₛₛ₁” and “Δₛₛ₂” as separate reference points, wherein each location on the line graph between these reference points is representative of an independent unique comparison “Δₛₛ”; and
referencing an observed blood flow measurement “F” to a location on the line graph with a “P_{diastolic}” to identify a “P” as an indication of the patient's blood pressure.

17. The method of claim 16 wherein there is a unique “Δₛₛ” at each location on the line graph between the different quantified “Δₛₛ”, and further wherein between successive “Δₛₛ” on the line graph the rate of change “ΔP” is not equal to the rate of change in “ΔF” with a new value for each unique “Δₛₛ”=(P±ΔP) and (F±ΔF).

18. The method of claim 16 wherein “P₁” and “P₂” are measured using a sphygmomanometer, and “Fₘₐₓ₁” and “Fₘₐₓ₂” are measured using an oximeter.

19. The method of claim 18 wherein “P_{systolic}” is measured near the beginning of the heart muscle cycle, while “P_{diastolic}” and “Fₘₐₓ” is measured near the end of the heart muscle cycle, wherein “P” and “Fₘₐₓ” have concurrence within a same heart muscle cycle, and further wherein different data sets are established with the patient respectively sitting, standing, and lying down.

20. The method of claim 19 wherein the line graph is created using the “P” and “Fₘₐₓ” values taken for successive quantified “Δₛₛ”, and wherein to account for “P” and “Fₘₐₓ” having an inverse relationship, a horizontal axis for the graph will show a decreasing value for “F” while a vertical axis for the graph will show an increasing value for “P”, and wherein each location on the resulting line graph represents a comparison “Δₛₛ” having unique values for “P” and “F”.
